(19) United States
(12) Patent Application Publication
(10) Pub. No.: US 2007/0202125 A1
(43) Pub. Date: Aug. 30, 2007

(54) Inhibiting rejection of a graft

(75) Inventor: Terry B. Strom, Brookline, MA (US)

Correspondence Address: FISH & RICHARDSON PC, P.O. BOX 1022, MINNEAPOLIS, MN, 55440-1022, US

(73) Assignee: Beth Israel Hospital Association, a Massachusetts corporation

(21) Appl. No.: 11/511,081

(22) Filed: Aug. 28, 2006

Related U.S. Application Data
(63) Continuation of application No. 10/228,873, filed on Aug. 26, 2002, now abandoned.
Application No. 10/228,873 is a continuation of application No. 08/417,077, filed on Apr. 5, 1995, now abandoned.

(51) Int. Cl. A61K 39/00

(52) U.S. Cl. 424/192.1; 424/184.1; 424/193.1

(57) ABSTRACT

Disclosed are methods for inhibit rejection of a graft in a patient. The methods involve treating the graft with a molecule which binds to a co-stimulatory protein of antigen-presenting cells. Useful molecules include chimeras having enzymatically inactive polypeptides bonded to polypeptides which bind to co-stimulatory proteins of antigen-presenting cells. Also disclosed, are chimeric molecules composed of lytic IgG Fc bonded to CD2, CD28, CD40L, or CTLA-4. In addition, disclosed are methods for inhibiting rejection of a graft in a patient; the methods involve treating the brain-dead, beating heart donor of the graft, prior to removal of the graft from the donor, to render the graft less susceptible to rejection by the patient.

### BACKGROUND OF THE INVENTION

This invention relates to inhibiting rejection of a graft in a patient.

T-cells play an important role in the rejection of allografts and xenografts (also referred to herein as “grafts”). Activation of T-cells bearing clonotypic receptors for donor alloantigen requires two distinct signals. The binding of a T-cell receptor to an alloantigen serves as one signal. The second signal, which is not delivered via the T-cell receptor, has been termed a co-stimulatory signal. The co-stimulatory signal is based on the interaction of ligands on the surfaces of antigen presenting cells (APCs) and T-cells (for a review, see Janeway et al., 1994, Cell 76: 275). For example, members of the B7 family of co-stimulatory proteins, including B7-1, B7-2, and B7-3, are expressed on APCs and interact with the CD28 T-cell surface protein. Engagement of the CD2 protein on T-cells with LFA-3 or CD48 on APCs also provides a co-stimulatory signal. After receiving both signal one and signal two, a T-cell proliferates and differentiates into an armed effector cell. T-cells that bind antigen without receiving a co-stimulatory signal are thought to undergo apoptosis or to become anergic (i.e., they fail to proliferate in response to antigenic rechallenge).

In a mixed lymphocyte culture (MLC), the T-cell proliferative response to alloantigen can be inhibited by blocking binding of B7 to CD28 (Tan et al., 1993, J. Exp. Med. 177: 165-173). In such an in vitro system, binding can be blocked in the presence of CTLA-4Ig, a chimeric immunoglobulin fusion protein incorporating the extracellular domain of CTLA-4. The extracellular domains of CTLA-4 and CD28 have considerable homology. CTLA-4 or CTLA-4Ig, however, binds B7 with higher affinity than does CD28. The systemic application of CTLA-4Ig promotes engraftment and can lead to tolerance of the graft when administered to recipient mice upon transplantation of pancreatic islet cells (Lenschow et al., Science, 1992, 257:789).

### SUMMARY OF THE INVENTION

I have found that rejection of a graft containing a cell which expresses a co-stimulatory protein(s) can be inhibited by treating (i.e., coating) the graft, in lieu of treating the recipient of the graft (i.e., the patient), to inhibit generation of a co-stimulatory signal and activation of host T-cells by the graft. Accordingly, in one aspect, the invention features inhibiting rejection of a graft containing a cell which expresses a co-stimulatory protein in a patient.(e.g., a human) involving treatment of the graft in the patient with a molecule, other than lytic CTLA-4/Fc, which binds to a co-stimulatory protein that is expressed upon antigen-presenting cells, thereby inhibiting activation of host T-cells by the graft. In embodiments of this aspect of the invention, the graft can also be treated ex vivo and/or in the donor (e.g., a brain-dead, beating-heart donor).

In a second aspect, the invention features a method for inhibiting rejection of a graft containing a cell that expresses a co-stimulatory protein in a patient, involving treating the graft outside of the patient with a molecule which binds to a co-stimulatory protein of antigen presenting cells, thereby inhibiting activation of host T-cells by the graft. The graft is treated ex vivo (i.e., in vitro) or, preferably, the graft is treated in a brain-dead, beating heart donor. If desired, the graft can be treated with a combination of methods. For example, the graft can be treated (1) in the brain-dead, beating-heart donor and ex vivo, (2) ex vivo and in the patient (e.g., by perfusing a chimeric molecule into the graft, with closure of the surgical anastomosis between the donor and the patient), (3) in the brain-dead, beating-heart donor and in the patient, or (4) in the brain-dead, beating-heart donor, ex vivo, and in the patient.

Suitable molecules for use in the first and second aspects of the invention include CTLA-4, CD28, CD40L (i.e., CD40 ligand), and/or CD2. Other suitable molecules include chimeric molecules that have (i) a first polypeptide which binds to a co-stimulatory protein of antigen-presenting cells bonded to (ii) a second polypeptide, the second polypeptide being one which is enzymatically inactive (e.g., non-lytic IgG heavy chains or portions thereof) in humans and which increases the circulating half-life of the first polypeptide by a factor of at least two. Where the graft is treated outside of the patient, monoclonal antibodies which specifically bind to co-stimulatory proteins of antigen-presenting cells can be used to treat the graft. These monoclonal antibodies can be identified by their ability to block the ectodomain of T-cell surface proteins from binding to co-stimulatory proteins on antigen-presenting cells. Suitable monoclonal antibodies include those which specifically bind to CD48, CD40, LFA-3, or a B7 protein such as B7-1, B7-2, or B7-3 (see, e.g., Gimmi et al., 1991, Proc. Nat'l. Acad. Sci. 88:6575-6579; Freeman et al., 1989, J. Immunol. 143:2714-2722; Boussiotis et al., 1993, Proc. Nat'l. Acad. Sci. 90:11059-11063; and Engel et al., 1994, Blood 84: 1402-1407).

In certain embodiments of the first and second aspects of the invention, the co-stimulatory protein is (1) a B7 protein, such as B7 -B1, B7-B2, B7-B3, or (2) CD48, (3) CD40, or (4) LFA-3. Where the molecule is a chimeric molecule of a first and second polypeptide, the first polypeptide of the chimera is one which binds to a co-stimulatory protein of antigen-presenting cells (e.g., CTLA-4, CD28, CD40L, or CD2). The second polypeptide is one which is enzymatically inactive in humans and which increases the circulating half-life of the first polypeptide by a factor of at least two. Examples of suitable second polypeptides are albumin and the Fc region of an IgG molecule or portions thereof which lack an IgG variable region of a heavy chain. Other useful second polypeptides include polypeptides that have enzymatic activity in an organism other than humans but which are enzymatically inactive in humans. For example, useful polypeptides include plant enzymes, porcine or rodent glycosyltransferases, and α-1,3 galactosyltransferases (see, e.g., Sandrin et al., 1993, Proc. Nat'l. Acad. Sci. 90:11391). In addition, mutated versions of polypeptides that normally have enzymatic activity in humans (e.g., enzymatically inactive human tissue plasminogen activator) can be used if the mutation(s) renders the polypeptide enzymatically inactive in humans.

Where the second polypeptide of the chimera is the Fc region of an IgG molecule, the Fc region can be either lytic or non-lytic (i.e., include a mutation which inhibits complement fixation and high affinity binding to the Fc receptor or a portion of the Fc region lacking the residues that (a) are necessary for activation of complement or (b) bind to the Fc receptors). A preferred class of chimeric molecules of the invention have non-lytic IgG Fc bonded to CD2, CTLA-4, CD28, or CD40L.

If desired, the second polypeptide of a chimeric molecule can include an IgG hinge region. In this embodiment, the IgG hinge region is positioned between the first polypeptide of the chimera (i.e., the polypeptide which binds to a co-stimulatory protein of antigen-presenting cells), and a half-life-increasing polypeptide (e.g., IgG Fc or albumin). If desired, the chimeric molecule can include a hinge region and an IgG Fc region while lacking the an Fc receptor binding site and/or a C'lq binding site. Where an IgG hinge region is employed, the IgG hinge region serves as a flexible polypeptide spacer, ensuring that the polypeptide which binds to a co-stimulatory protein is not physically constrained by the half-life-increasing polypeptide. As an alternative to using an IgG hinge region, a flexible polypeptide spacer, as defined herein, can be used. Using conventional molecular biology techniques, such a polypeptide spacer can be inserted between the half-life-increasing polypeptide and the protein which binds to a co-stimulatory protein.

If desired, the graft can be treated with a combination of molecules. For example, the graft can be treated with CD28 or CTLA-4 ex vivo and then with a lytic CD2/Fc chimera in the patient. In preferred combinations, the graft is treated with a CTLA-4/Fc chimera and with a CD2/Fc chimera, either simultaneously or sequentially.

In another aspect, the invention features chimeric molecules having a first polypeptide which includes CD2, CTLA-4, CD28, or CD40L covalently bonded to a second polypeptide which includes non-lytic IgG Fc. Preferred molecules include IgG Fc covalently bonded to a hinge region which is covalently bonded to CD2, CTLA-4, CD40L, or CD28. The aforementioned molecules are useful in inhibiting rejection of a graft in the methods described herein.

The invention also features inhibiting rejection of a graft in a patient, involving treating the brain-dead, beating-heart donor of the graft, prior to removal of the graft from the donor, to render the graft less susceptible to posttransplantation rejection by the patient. In a preferred embodiment, treatment involves modifying, eliminating, or masking a cell-surface protein of the graft. The cell-surface protein can be one which is capable of causing a co-stimulatory signal in T-lymphocytes in the patient (e.g., a co-stimulatory protein such as a B7 protein), or the cell-surface protein can be any antigen which is capable of causing a T-lymphocyte-mediated response in the patient (e.g., ICAM-1). The cell-surface antigen or co-stimulatory protein can be masked by treating the graft with a non-lytic masking agent which includes an antibody F(ab′)₂ fragment which is capable of forming a complex with an antigen or co-stimulatory protein on the cell. If desired, a cell bearing a co-stimulatory protein can be lysed with a chimeric molecule which has (i) a polypeptide which binds to a co-stimulatory protein fused to (ii) a polypeptide which has a lytic Fc region of an IgG molecule and which lacks an IgG heavy chain variable region.

By “graft” is meant any cell, tissue, or organ (e.g., islet cells, and kidney, heart, liver, lung, brain, and muscle tissues) transplanted from one individual (e.g., a mammal such as a human) to another.

By IgG “Fc” region is meant a naturally-occurring or synthetic polypeptide homologous to the IgG C-terminal domain that is produced upon papain digestion of IgG. IgG Fc has a molecular weight of approximately 50 kD. In the molecules of the invention, the entire Fc region can be used, or only a half-life enhancing portion. In addition, many modifications in amino acid sequence are acceptable, as native activity is not in all cases necessary or desired.

By “non-lytic” IgG Fc is meant an IgG Fc region which lacks a high affinity Fc receptor binding site and which lacks a C'lq binding site. The high affinity Fc receptor binding site includes the Leu residue at position 235 of IgG Fc; the Fc receptor binding site can be functionally destroyed by mutating or deleting Leu 235. For example, substitution of Glu for Leu 235 inhibits the ability of the Fc region to bind the high affinity Fc receptor. The C'lq binding site can be functionally destroyed by mutating or deleting the Glu 318, Lys 320, and Lys 322 residues of IgG1. For example, substitution of Ala residues for Glu 318, Lys 320, and Lys 322 renders IgG1 Fc unable to direct ADCC.

By “lytic” IgG Fc is meant an IgG Fc region which has a high affinity Fc receptor binding site and a C'lq binding site. The high affinity Fc receptor binding site includes the Leu residue at position 235 of the IgG Fc. The C'lq binding site includes the Glu 318, Lys 320, and Lys 322 residues of IgG1. Lytic IgG Fc has wild-type residues or conservative amino acid substitutions at these binding sites. Lytic IgG Fc can target cells for antibody dependent cellular cytotoxicity (ADCC) or complement directed cytolysis (CDC).

By IgG “hinge” region is meant a polypeptide homologous to the portion of a naturally-occurring IgG which includes the cysteine residues at which the disulfide bonds linking the two heavy chains of the immunoglobulin form. For IgG1, the hinge region also includes the cysteine residues at which the disulfide bonds linking the γ1 and light chains form. The hinge region is approximately 13-18 amino acids in length in IgG1, IgG2, and IGg4; in IgG3, the hinge region is approximately 65 amino acids in length.

By polypeptide “spacer” is meant a polypeptide which, when placed between the half-life-increasing polypeptide and the polypeptide which binds to a co-stimulatory protein of antigen-presenting cells, possesses an amino acid residue with a normalized B value (Bₙₒᵣₘ; a measure of flexibility) of 1.000 or greater, preferably of 1.125 or greater, and, most preferably of 1.135 or greater (see, e.g., Karplus et al., 1985, Naturwissenschaften 72:212). Amino acids which are commonly known to be flexible include glutamic acid, glutamine, threonine, lysine, serine, glycine, proline, aspartic acid, asparagine, and arginine.

The invention provides a method for inhibiting rejection of a graft; accordingly, the invention is useful for protecting the graft from rejection and promoting tolerance of a transplanted cell, organ, or tissue. One advantage of the invention is that it obviates systemic immunosuppression of the patient. Treating the graft outside of the patient blocks co-stimulation by donor graft antigens and leaves normal protective immune responses to non-graft antigens unimpaired.

Other features and advantages of the invention will be apparent from the following description of the preferred embodiments thereof, and from the claims.

### DETAILED DESCRIPTION

The drawings will first be briefly described.

Drawings

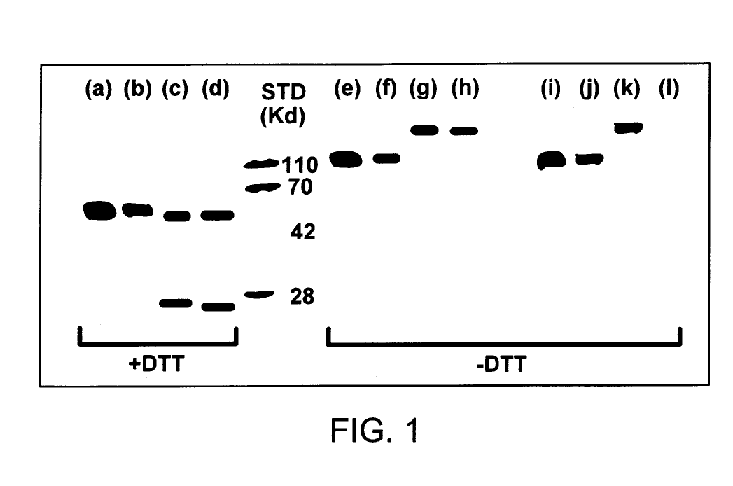

FIG. 1 is a reproduction of a polyacrylamide gel used to confirm the size and isotope specificity of lytic (L) and non-lytic (NL) mCTLA-4/Fc. Affinity-purified protein was characterized by Laemmeli gel electrophoresis under reducing (+DTT) and non-reducing (−DTT) conditions. Protein in lanes (a)-(h) was visualized by coomassie blue staining. Protein in lanes (i)-(l) was stained with rat anti-mouse IgG2a and detected by Western blot to confirm the IgG2a isotope specificity. Lytic CTLA-4/Fc was loaded in lanes (a), (e), and (i); non-lytic CTLA-4/Fc was loaded in lanes (b), (f), and (j); mIgG2a was loaded in lanes (c), (g), and (k); and mIgG3 was loaded in lanes (d), (h), and (l). Blots were scanned using Scanjet II software (Hewlett Packard, Greeley, Col.).

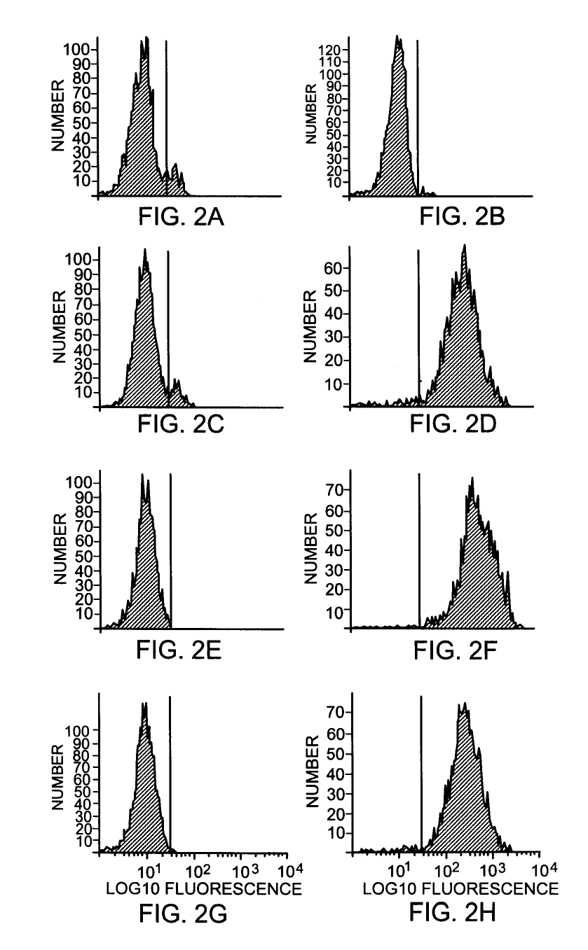

FIGS. 2A-H are a series of FACS profiles confirming binding of B7-1 by (L) and (NL) mCTLA-4/Fc. CHO cells transfected with vector alone (FIGS. 2A, 2C, 2E, and 2G) or B7-1-transfected CHO cells (2.5×10⁵) were incubated with 10 μg/ml of mIgG2a (negative control; FIGS. 2A and 2B), 100 μg/ml of an anti-B7-1 mAb (positive control; FIGS. 2C and 2D), 10 μg/ml of (L) mCTLA-4/Fc (FIGS. 2E and 2F), or 10 μg/ml of (NL) mCTLA-4/Fc (FIGS. 2G and 2H).

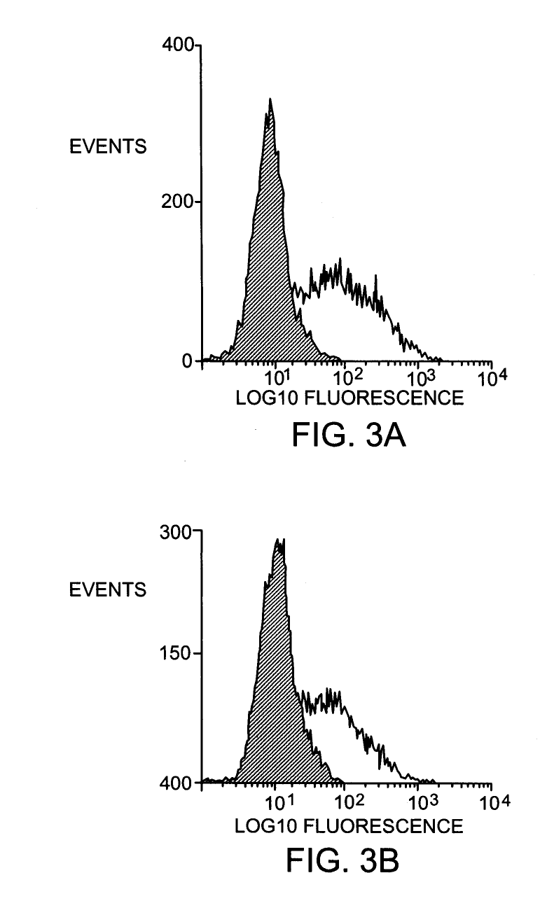

FIGS. 3A-B are two FACS profiles indicating that (L), but not (NL), mCTLA-4/Fc binds the high affinity FcγRI. For FIG. 3A, FcγRI-transfected CHO cells (2.5×10⁵) were incubated with 10 μg/ml of mIgG2a (positive control, open profile) or media alone (negative control, solid profile). For FIG. 3B, FcγRI-transfected CHO cells were incubated with 10 μg/ml of (L) mCTLA-4/Fc (open profile) or (NL) mCTLA-4/Fc (solid profile).

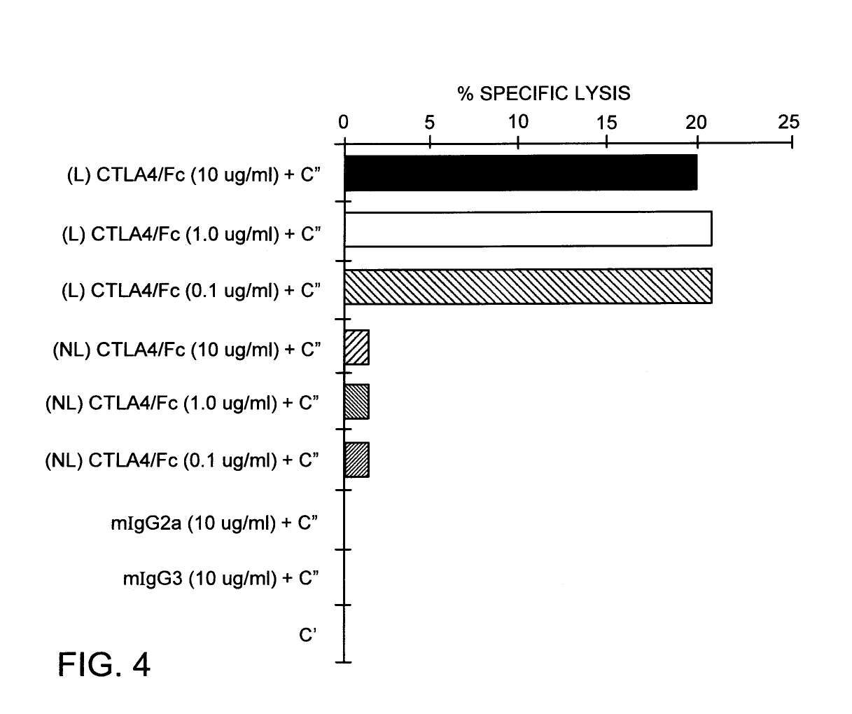

FIG. 4 is a histogram indicating that (L), but not (NL) mCTLA-4/Fc, lyses cells expressing B7-1. B7-1-transfected CHO cells (10⁶) that were labeled with 100 μCi ⁵¹Cr were incubated with various concentrations of (L) or (NL) mCTLA-4/Fc and rabbit low tox complement. Cells incubated with mIgG2a and complement, mIgG3 and complement, or complement alone served to define non-specific lysis.

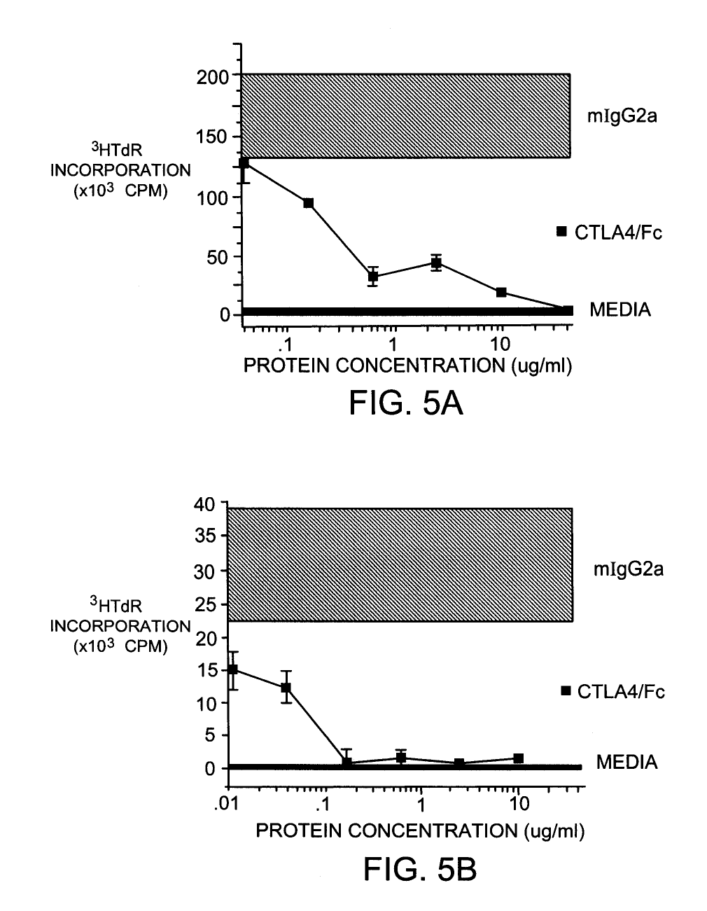

FIGS. 5A-B are graphs showing that mCTLA-4 inhibits the proliferation of unfractionated spleen cell cultures. For FIG. 5A, Con A-stimulated B6AF1 spleen cells were incubated with varying concentrations of (L) mCTLA-4/Fc, control mIgG2a monoclonal antibodies, or media alone. The data presented in FIG. 5B were obtained from a mixed lymphocyte culture. For the mixed lymphocyte culture, aliquots of DBA2/J (H-2^{d}) responder cells (10⁵ cells/well) that had been preincubated with serial dilutions of (L) mCTLA-4/Fc were stimulated with irradiated (3000 rad) C57B1/6 (H-2^{b}) spleen cells (2×10⁵ cells/well) harvested on day 5 of culture.

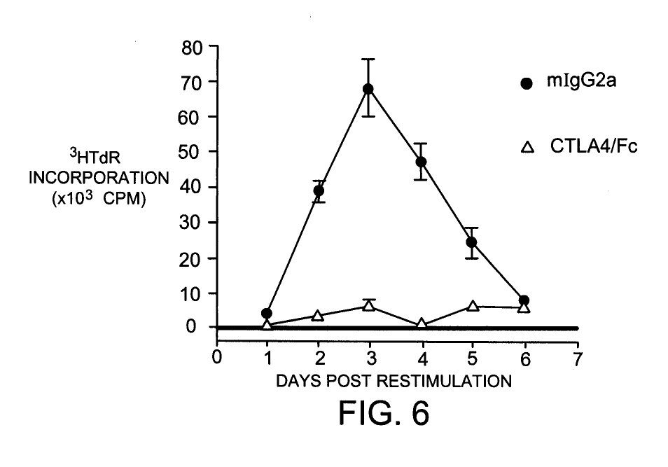

FIG. 6 is a graph showing that the application of mCTLA-4/Fc to a primary MLC induces hyporesponsiveness of responder strain cells that is evident upon re-stimulation of an MLC with stimulator strain cells. MLCs were established using 2×10⁷ spleen cells at a 1:1 responder: stimulator ratio in 6-well culture plates in the presence of 10 μg/ml mCTLA-4/Fc or mIgG2a. DBA2/J cells were washed extensively on day 7, cultured for another 3 days in medium without mCTLA-4/Fc or mIgG2a, and then re-stimulated with irradiated C56B1/6 spleen cells. Aliquots were harvested daily on days 1 through 7.

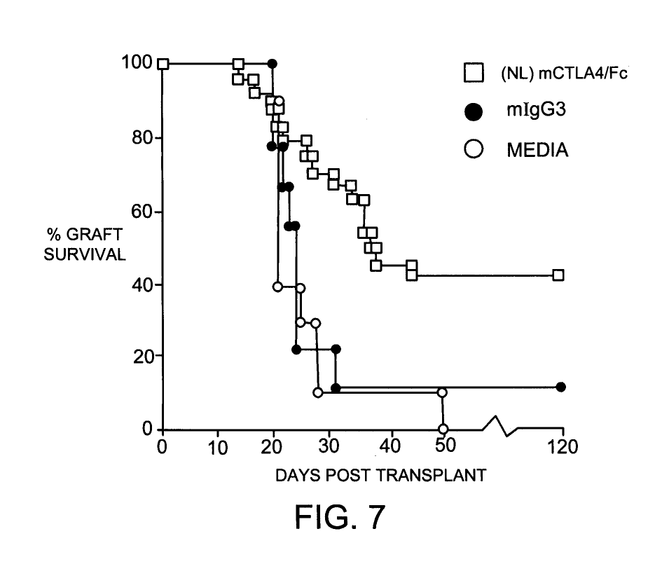

FIG. 7 is a graph indicating that islet cell allograft treatment with (NL) CTLA-4/Fc prolongs engraftment. Fresh islet cell isolates harvested from DBA/2J mice were incubated for 1 hour prior to implantation with either media alone, 10 μg/ml mIgG3 (control protein), or 10 μg/ml (NL) mCTLA-4/Fc. Subsequently, 300-400 islets were injected under the left renal capsule of streptozotocin-treated diabetic B6AF1 recipients, and graft function was followed by monitoring blood glucose levels.

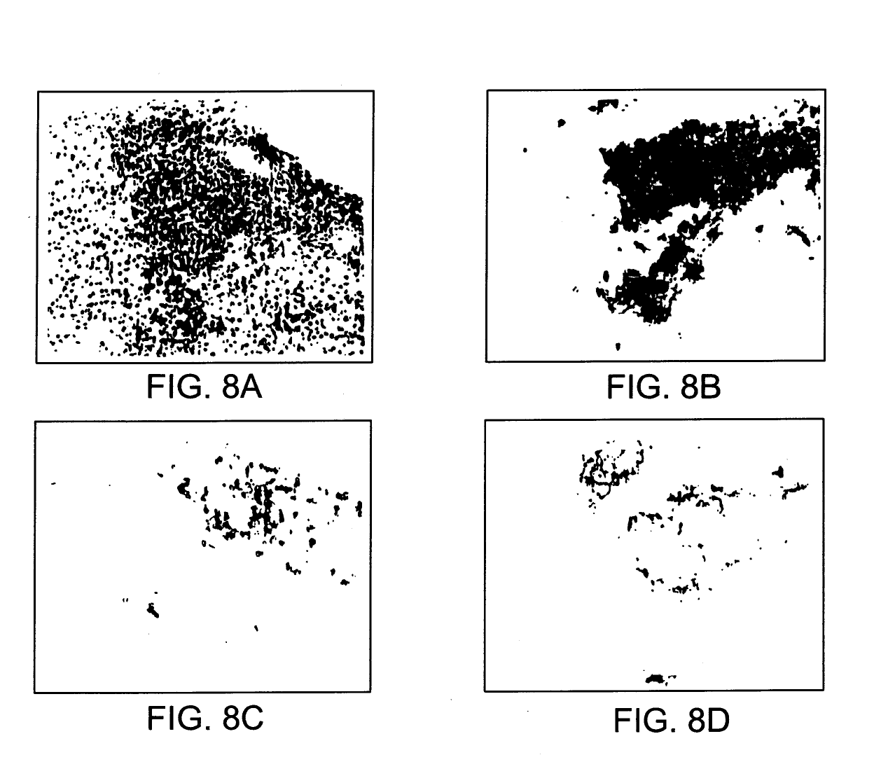

FIGS. 8A-D are a series of photographs obtained during a histologic analysis of islet grafts in tolerant hosts. FIG. 8A is a photograph indicating that tolerance to an islet allograft treated with (NL) CTLA-4/Fc is not synonymous with the absence of an allograft response (hematoxylin and eosin staining; 200×); M, mononuclear cell infiltrate; S, intact islet. FIG. 8B is a photograph showing cells stained with rat anti-mouse CD4 monoclonal Ab (200×), and FIG. 8C is a photograph indicating that cells stained with rat anti-mouse CD8⁺ monoclonal Ab (200×) surround, but do not invade, the islet allografts in tolerant mCTLA-4/Fc treated hosts. FIG. 8D is a photograph displaying the results of immunohistology of a graft incubated with the exclusion of a primary antibody (200×).

Before providing detailed working examples of the invention, some of the preferred molecules of the invention are described in detail to serve as examples of molecules that can be used in the invention.

CTLA-4/Fc: Useful CTLA-4/Fc chimeric proteins include proteins having the extracellular region of CTLA-4 fused to the CH2 and CH3 portions of IgG heavy chain, with or without the hinge region. Such molecules can be produced with standard recombinant DNA techniques and conventional protein purification methods. Protein purification methods can employ protein A to form an affinity complex with the Fc portion of the molecule. Alternatively, or in addition, anti-CTLA-4 antibodies can be used to bind the CTLA-4 portion of the chimera. An example of a useful protein is CTLA-4Ig, described by Linsley et al. (J. Exp. Med., 1991, 174:561). Preferably, CTLA-4 and IgG are derived from human sources. Less preferably, CTLA-4 and/or IgG can be derived from non-human sources, such as mice. The portion of CTLA-4 to be used in the invention should be sufficient to bind to at least one of the co-stimulatory proteins of APCs; such portions of proteins can be identified with conventional methods. For example, useful portions of proteins can be identified by their ability to bind to B7⁺ CHO cells as determined by FACS analysis (see, e.g., Linsley et al., J. Exp. Med., 1991, 174:561).

If desired, the Fc region can be mutated to diminish its ability to fix complement and/or bind the Fc receptor with high affinity; this renders the chimeric molecule non-lytic. Thus, a non-lytic chimeric molecule can be created with standard mutagenesis methods by mutating the high affinity Fc receptor binding site and the C'lq binding site of the Fc portion of the chimeric molecule. For example, substitution of Ala residues for Glu 318, Lys 320, and Lys 322 renders IgG1 Fc unable to direct ADCC, and substitution of Glu for Leu 235 inhibits the ability of the Fc region to bind the high affinity Fc receptor (see e.g., Morrison et al., The Immunologist, 1994, 2:119 and Brekke et al., The Immunologist, 1994, 2:125).

CD2/Fc, CD40L/Fc, and CD28/Fc: Also useful in the invention are chimeric molecules composed of CD2, CD40L, or CD28 bonded to the Fc region of IgG. These molecules which block B7-mediated co-stimulatory signals, can be made by employing standard molecular biology techniques to fuse all or a portion of CD2, CD40L, or CD28 to the CH2 and CH3 regions of IgG Fc, with or without the hinge region. Where a portion of CD2, CD40L, or CD28 is employed, the portion should be sufficient to bind to a co-stimulatory protein, as determined with standard methods. The chimeric proteins can be synthesized by employing standard methods for protein expression. In addition, the molecules can be purified with art-recognized techniques. For example, a protein A column can be utilized to affinity-purify the chimeric molecules. In addition, antibodies directed against the CD2, CD40L, or CD28 portion of the chimera can be used.

There now follows a description of some of the additional parameters of the invention.

Chimeric Proteins: Conventional molecular biology techniques can be used to produce chimeric proteins having a first polypeptide which binds to a co-stimulatory protein bonded to a second polypeptide, which is an enzymatically inactive polypeptide (e.g., a lytic or non-lytic Fc region of IgG). Numerous polypeptides are suitable for use as enzymatically inactive polypeptides in the invention. Examples include the Fc region of IgG in the absence of a variable region of a heavy chain, albumin (e.g., human serum albumin), transferrin, enzymes that are not active in humans, and other proteins having a long circulating half-life. Preferably, the enzymatically inactive polypeptide has a molecular weight of at least 10 kD; a net neutral charge at pH 6.8; a globular tertiary structure, human origin; and no ability to bind to cell surface proteins other than the co-stimulatory protein to which the first polypeptide of the chimera binds (e.g., a B7 protein). Where the enzymatically inactive polypeptide is IgG, preferably, the IgG portion is glycosylated.

Preferably, the enzymatically inactive polypeptide used in the production of the chimeric protein (e.g., IgG Fc) has, by itself, an in vivo circulating half-life greater than that of the polypeptide (i.e., the first polypeptide of the chimera) which binds the co-stimulatory protein. More preferably, the half-life of the chimeric protein is at least 2 times that of the first polypeptide alone; most preferably, the half-life of the chimeric protein is at least 10 times that of the first polypeptide alone.

The circulating half-life of the chimeric protein can be measured in an ELISA of a sample of serum obtained from a mammal treated with the chimeric protein. In such an ELISA, antibodies directed against the first polypeptide of the chimera (i.e., the polypeptide which binds the co-stimulatory protein) can be used as the capture antibodies, and antibodies directed against the enzymatically inactive protein can be used as the detection antibodies, allowing detection of only the chimeric protein in a sample. Conventional methods for performing ELISAs can be used.

An important feature of the molecules used in the invention is the ability to bind to at least one of the co-stimulatory proteins (e.g., a B7 protein, LFA-3, or CD48). The ability of a molecule to bind to a B7 protein can be assayed with conventional methods, for example, with B7⁺ cells (for a detailed example, see below). Accordingly, the molecule which binds to the co-stimulatory protein of APCs can be a portion of a naturally-occurring protein, provided that the portion which is used has the ability to bind to a co-stimulatory protein. Similarly, mutated proteins can be used in the creation of useful molecules, provided that the molecule can bind to a co-stimulatory protein. It is not necessary that the activity of the chimeric protein be identical to the activity of the first polypeptide of the chimera alone. For example, the chimeric protein may bind the co-stimulatory protein with more or less avidity than does the first polypeptide of the chimera alone.

If desired, the enzymatically inactive polypeptide can include an IgG hinge region positioned such that the chimeric protein has a first polypeptide (i.e., the polypeptide which binds a co-stimulatory protein) bonded to an IgG hinge region, with the hinge region bonded to a longevity-increasing polypeptide (e.g., an albumin or the CH2 and CH3 regions of an IgG). A person skilled in molecular biology can readily produce such molecules from an IgG2a-secreting hybridoma (e.g., HB129), other eukaryotic cells, or baculovirus systems.

Treating the Graft: Ex vivo treatment of the graft can be accomplished with standard techniques (including the use of infusion pumps and syringes) for perfusing fluids into organs, cells, or tissues. If desired, conventional immunohistology methods can be used to assay the degree to which the graft is coated with the molecule which binds the co-stimulatory protein (see, e.g., Brewer et al., 1989, The Lancet 2:935). Generally, the concentration of the molecule will be 0.1 to 10 mg/ml; preferably, the concentration is 0.5 to 2 mg/ml. If desired, the graft can simply be immersed in a solution of the desired chimeric molecule(s) (e.g., CTLA-4/FC) and a physiologically acceptable carrier (e.g., saline). Generally, the graft will be incubated for 30 minutes to 1 week; preferably, where intact organs are used, the intact organ is incubated for approximately 30 minutes , and where cultured cells are used, cultured cells are incubated for several days. Generally, for immersion, the concentration of the molecule which binds to a co-stimulatory protein will be 0.1 mg/ml to 10 mg/ml.

Treatment of the graft in a patient or brain dead, beating-heart donor can be accomplished by simply injecting (e.g., intraperitoneally, intravenously, or intra-arterially) or gradually infusing a solution of the co-stimulatory protein-binding molecule and a physiologically acceptable carrier into the donor. For example, the solution can be delivered into a blood vessel of the donor via one of the intravenous lines typically already present in such patients or donors. Generally, the amount of the co-stimulatory protein-binding molecule to be injected will be 1.0 mg to 500 mg, preferably, 5 mg to 50 mg at a concentration of 0.1 μg/ml to 5 mg/ml. When treating grafts in brain dead, beating-heart donors, 0.1 to 1.0 hour of incubation prior to removal of the graft is generally sufficient for inhibiting rejection of the graft in a patient.

Inhibition of Graft Reflection by Treating the Graft in the Donor.

More generally, the invention features any treatment of a graft prior to its removal from a brain-dead, beating-heart donor to inhibit subsequent rejection of the graft in a patient (i.e., recipient). For example, rejection of the graft can be inhibited by modifying, eliminating, or masking a cell-surface protein of the graft. The cell-surface protein can be an antigen which, when present on the surface of a cell of the graft, is capable of causing a T-lymphocyte-mediated response in the patient. Similarly, a co-stimulatory protein of the graft can be masked, modified, or eliminated to inhibit the generation of a co-stimulatory signal in T-lymphocytes in the patient. Known masking agents include F(ab′)₂ fragments of antibodies directed against co-stimulatory proteins (e.g., a B7 protein) or donor cell antigens (e.g., HLA class 1 antigens). Alternatively, rejection can be inhibited by masking an antigen on the surface of the graft with the use of a soluble host T-cell receptor(s) (i.e., the patient's T-cell receptor) which binds an antigenic site(s) on the graft that would otherwise interact with the patient's T-cells in vivo. Also useful are synthetic organic molecules which mimic the antigen-binding properties of T-cell receptors. If desired, the cell bearing a co-stimulatory protein can be lysed with a chimeric molecule which has (i) a polypeptide which binds. to a co-stimulatory protein of antigen-presenting cells fused to (ii) a polypeptide which has a lytic Fc region of an IgG molecule and which lacks a variable region of an IgG heavy chain.

A detailed discussion of methods for masking, eliminating, or modifying a cell-surface antigen is provided in U.S. Pat. No. 5,283,058, hereby incorporated by reference. The methods described therein are also appropriate for masking, eliminating, or modifying a cell-surface co-stimulatory protein. In this aspect of the invention, the graft is treated in the brain-dead, beating-heart donor by perfusion of a solution of the desired masking, eliminating, or modifying agent and a physiologically acceptable carrier into the graft. The graft can also be treated by injecting into the donor (e.g., intraperitoneally, intravenously, or intra-arterially) a solution of a molecule which binds to or co-stimulatory protein or an antigen of antigen-presenting cells.

There now follows a brief discussion of some parameters of the example.

Animals: Six to eight week old male B6AF1, DBA/2J, and C57BL/6 mice were obtained from the Jackson Laboratory (Bar Harbor, ME) and housed under standard conditions both before and after transplantation.

Monoclonal Antibodies: The following monoclonal antibodies were used: rat anti-mouse IgG2a (Pharmingen, San Diego, Calif.), rat anti-mouse IgG2A-horseradish peroxidase (HRPO) (Pharmigen), FITC-labeled goat anti-mouse IgG (Sigma, St. Louis, Mo.), rat anti-mouse CD4 (Pharmigen), rat anti-mouse CD8 (Pharmigen), biotinylated rabbit anti-rat mAb (Vector, Burlingame, Calif.), hamster anti-mouse B7-1 16-10 A1, FITC-labeled rabbit anti-hamster IgG (Pierce, Rockford, Ill.), and mouse IgG2a (kappa) and IgG3 (kappa) hybridoma proteins (Cappel, West Chester, Pa.).

Cell Lines: The following cell lines were used: murine IgG2a-secreting hybridoma 116-13.1 (American Type Culture Collection (ATCC), Rockville, Md.), CHO-KI (ATCC), CHO cells transfected with human FcγRI cDNA, CHO cells transfected with DNA encoding mouse B7-1, and CHO cells transfected with a CMV-based vector alone.

Cell Cultures: Cell culture reagents, unless otherwise stated, were obtained from Gibco BRL (Grand Island, N.Y.). Cells were grown in complete RPMI 1640, i.e., RPMI supplemented with L-glutamine, 10% heat-inactivated fetal calf serum (FCS), 10 mM HEPES, 0.1 mM non-essential amino acids, 1 mM sodium pyruvate, 5×10⁻⁵ M 2-mercaptoethanol, 100 U/ml penicillin, and 100 μmg/ml streptomycin. CHO-KI transfectants were maintained in DMEM with 5% FCS, 100 U/ml penicillin, and 100 mg/ml streptomycin. Transfected cell lines were cultured in Ultraculture (Bio Whittaker, Walkersville, Md.) serum-free media supplemented with L-glutamine, penicillin, and streptomycin.

Plasmids: The examples employ the murine CTLA-4 cDNA plasmid F41F4 (Brunet et al., 1987, Nature 328:267). The eukaryotic expression vector Rc/CMV (Invitrogen, San Diego, Calif.) was modified by deletion of all three BamHI sites and its unique ApaI site. The PCR II vector (Invitrogen) was used for TA cloning of cDNA amplified by the polymerase chain reaction (PCR).

Genetic Constructs: Total RNA was purified, on a cesium chloride gradient, from the murine IgG2a-secreting hybridoma 116-13.1 and then reverse-transcribed to cDNA using oligo-dT₁₂₋₁₈ primers and M-MLV reverse transcriptase. The region of the Fcγ2a cDNA encoding the hinge, CH2, and CH3 regions of the heavy chain was then amplified by PCR using oligonucleotides designed to append unique BamHI and XbaI restriction sites onto the 5′ and 3′ ends, respectively, of the Fcγ2a CDNA fragment. The CDNA PCR product was digested with BamHI and XbaI restriction enzymes and gel-purified in preparation for ligation.

A 503 bp fragment of the murine CTLA-4 CDNA plasmid F41F4, encoding the leader and extracellular domains of CTLA-4, was amplified by PCR using oligonucleotide primers designed to append unique NotI and BamHI restriction sites onto the 5′ and 3′ ends, respectively, of this cDNA. The amplified cDNA was then cloned into the PCR II vector, excised using NotI and BamHI, and gel-purified. Subsequently, the CTLA-4 cDNA, the previously-prepared Fcy2a cDNA, and the cDNA of the modified Rc/CMV vector opened with NotI and XbaI at the cloning site were mixed and then ligated using T4 DNA ligase. The correct reading frame at the junction of the CTLA-4 and Fc cDNAs was confirmed by DNA sequencing.

PCR-assisted site-directed mutagenesis of the Fcγ2a cassette was employed to render non-functional (a) the high affinity FcγRI receptor binding site by substituting Glu for Leu 235 (see, e.g., Duncan, et al., 1988, Nature 332:563) and (b) the C'lq binding site, by substituting Glu 318, Lys 320, and Lys 322 with Ala residues (see e.g., Duncan, et al., 1988, Nature 332:738). The mutations were confirmed by DNA sequencing. Subsequent expression of the mutated CTLA-4/Fc construct results in a murine CTLA-4/Fc chimeric molecules without ADCC and CDC activity (i.e., non-lytic (NL) mCTLA-4/Fc).

mCTLA-4/Fc Expression and Purification: To achieve stable expression of CTLA-4/Fc in CHO-K1 cells, 20 μg of the murine CTLA-4/Fc plasmid construct was linearized by PvuI digestion and electroporated into 10⁷ CHO-K1 cells. Transformed CHO-K1 cells were selected with 1 mg/ml of G418 and subsequently cloned by limiting dilution. Established cell lines were then screened for mCTLA-4/Fc production with an ELISA that was specific for murine IgG2a. Clones were cultured in serum free media for 12 days. The supernatant was size-filtered (through a 0.2 μm pore), and Tris (pH 8.0) was added to the supernatant to a final concentration of 50 mM. The supernatant was then passed over a protein A sepharose column (Pharmacia) that had been equilibrated with 0.05 M TBS (pH 8.0), and mCTLA-4/Fc was eluted with 0.04 M sodium citrate (pH 4.5). Eluted fractions were immediately buffered to a pH of 7.4 by the addition of 0.1 volumes of 1 M Tris (pH 8.0). The fractions with the greatest absorbance at 280 nm were then pooled and dialyzed against PBS overnight at 4° C.

In Vitro Characterization of Lytic and Non-lytic mCTLA-4/Fc.

Confirmation of Size and Isotope Specificity: Affinity purified proteins were characterized by Laemmeli gel electrophoresis under reducing (+DTT) and non-reducing (−DTT) conditions. After the proteins were transferred to a nylon membrane (Immobilon-P, Millipore, Bedford, Mass.), the proteins were (a) visualized by coomassie blue staining and (b) analyzed by Western blot to confirm the IgG2a isotope specificity. Western blot analysis employed rat anti-mouse IgG2a as the primary antibody and a biotinylated rabbit anti-rat mAb as the secondary antibody. The complex was visualized with avidin-HRPO complex (Vector), using 3′, 3′-diaminobenzidine for detection of enzyme activity.

Coomassie blue staining revealed a single protein band at the expected molecular size of ˜55 kD (FIG. 1, lanes a and b). The murine IgG2a and mIgG3 control proteins each migrated as two protein bands of 25 and 50 kD, reflecting the kappa light chain and IgG2a heavy chain (FIG. 1, lanes c and d). Under non-reducing conditions (−DTT), (L) and (NL) mCTLA-4/Fc migrated as a single band with a molecular size of ˜110 kD, consistent with the formation of homodimers (FIG. 1, lanes e and f). The specific binding of a rat anti-mouse IgG2a mAb to mCTLA-4/Fc (FIG. 1 lanes i and j) confirmed the isotype specificity of the Fc portion of the chimeric proteins.

Confirmation of B7-1 binding: CHO cells (2.5×10⁵) transfected with B7-1 DNA were incubated at 4° C. with saturating concentrations (10 μg/ml) of (L) or (NL) mCTLA-4/Fc or 10 μg/ml of mIgG2a (negative control), washed twice, and then incubated with a 1:125 dilution of FITC-conjugated goat anti-mouse IgG mAb. To confirm the B7-1 surface expression of the transfected CHO cells (positive control), the cells were incubated at 4° C. with saturating concentrations (100 μg/ml) of hamster anti-mouse B7-1 mAb, washed twice, and then incubated with a 1:60 dilution of FITC-conjugated rabbit anti-hamster Ab. The cells were fixed in 1% formaldehyde and subsequently analyzed with a FACStar PLUS cell sorter (Becton Dickinson, Franklin Lakes, N.J.).

Staining of transfected CHO cells with the anti-mB7-1 monoclonal Ab 16-10 A1 and detection by FACS analysis indicated that CHO cells transfected with the full-length mouse B7 -B1 cDNA expressed high levels of mouse B7 -B1 (FIG. 2D). B7-negative CHO cells, transfected with the vector alone, served as a negative control (FIGS. 2A, 2C, 2E, and 2G). The difference between the FACS profiles of B7-1-transfected cells and control cells demonstrated that (L) and (NL) mCTLA-4/Fc bind to B7-1-transfected CHO cells (FIGS. 2E and 2G). In contrast, the isotype control (mIgG2a) did not bind to either the B7-negative CHO cells or B7-1-transfected CHO cells (FIGS. 2A and 2B).

Assessment of FcγRI binding: FcγRI-transfected CHO cells (2.5×10⁵) were incubated at 4° C. with saturating concentrations (10 μg/ml) of (L) or (NL) mCTLA-4/Fc or 10 μg/ml of mIgG2a (positive control), washed twice, and then incubated with a 1:125 dilution of FITC-conjugated goat anti-mouse IgG mAb. Cells that were incubated with media alone then incubated with a 1:125 dilution of FITC-conjugated goat anti-mouse IgC mAb served as a negative control. Cells were fixed in 1% formaldehyde and subsequently analyzed with a FACStar PLUS cell sorter. Lytic CTLA-4/Fc readily bound to FcγRI-transfected CHO cells (FIG. 3B, solid profile). Murine IgG2a, which bound to FcγRI+target cells in a similar manner to binding of (L) mCTLA-4/Fc, served as a positive control (FIG. 3A, open profile).

Complement Directed Cytotoxicity Assay: B7-1-transfected CHO cells (10⁶) were labeled with 100 μCi ⁵¹Cr, washed three times, distributed to a density of 10⁴ cells/well in flat-bottom microtiter plates, then incubated at 37° C. for 45 minutes with various dilutions of (L) or (NL) mCTLA-4/Fc and rabbit low tox complement (Cedarlane, Hornby, ONT, Canada) at a dilution of 1:10. The amount of ⁵¹Cr released by the cells into 100 μl aliquots of the culture supernatant was measured in a gamma counter. The maximum amount of ⁵¹Cr released was determined by lysis of ⁵¹Cr-labeled cells with Nonidet P-40. The percent specific lysis was calculated according to the formula: % specific lysis=(experimental cpm−background cpm)/(total release cpm−background cpm)×100. All experiments were performed in triplicate. In the presence of complement and (L) mCTLA-4/Fc, 20-21% specific lysis of B7-1-transfected CHO cells was detected (FIG. 4). In contrast, the presence of complement and (NL) mCTLA-4/Fc induced only a 1% specific lysis of B7 -B1 CHO cells (FIG. 4). Complement alone, mIgG2a and complement, or mIgG2 and complement were each ineffective in directing lysis of B7-1⁺ target cells (FIG. 4). These data indicate that (NL) CTLA-4/Fc does not target cells for CDC.

Assessment of Anti-proliferative Activity: To ascertain whether the mCTLA-4/Fc chimeric molecule was able to block murine T-cell activation, I examined the effect of CTLA-4/Fc in two in vitro systems of T-cell activation. In the first system, the in vitro immunosuppressive potential of mCTLA-4/Fc was tested in a concanavalin A (Con A) driven proliferation system in which APCs provide important co-stimulatory signals (Mueller et al., 1989, Annu. Rev. Immunol. 7:445). For Con A activation of unfractionated spleen cells, B6AF1 spleen cells were prepared by mincing a spleen between two glass slides. After washing the cells, red blood cells were lysed by exposing them to Tris-ammonium chloride buffer for 5 minutes at room temperature, and the mixture was then washed. TRYPAN BLUE™ (C₃₄H₂₄N₆O₁₄S₄Na₄) staining of the cells indicated that cell viability exceeded 90%. Following incubation with (L) mCTLA-4/Fc or control mIgG2a monoclonal Ab in 1:4 serial dilutions for 1 hour, 3×10⁵ spleen cells were cultured in flat-bottom 96-well microtiter plates in quadruplicate samples for 48 hours in a final volume of 200 μl. Proliferation was estimated by pulsing the cultures 6 hours before termination with 1 μCi/well [³H]thymidine, and [³H]thymidine incorporation was measured with a liquid scintillation counter.

The blockade of B7 sites with (L) mCTLA-4/Fc (FIG. 5A) and (NL) mCTLA-4/Fc (data not shown), but not control IgG2a, produced a dose-dependent anti-proliferative effect. I also tested the effect of (L) mCTLA-4/Fc on allogeneic MLCs. Proliferation, as estimated by [³H]thymidine incorporation on day 5 of culture, was markedly inhibited by (L) mCTLA-4/Fc (FIG. 5B). On a per dose basis, the MLC was more sensitive to the inhibitory effects of mCTLA-4/Fc than were Con A cultures.

Interference with the CD28-pathway during T-cell priming results in antigen-specific hyporesponsiveness upon secondary re-stimulation (Tan et al., 1993, J. Exp. Med. 177:165). In the second system, MLCs were used to determine whether mCTLA-4/Fc exerts similar long lasting effects on secondary murine T-cell responses. For the primary MLCs, 10⁵ DBA2/J (H-2d) responder cells were preincubated in serial dilutions of (L) mCTLA-4/Fc for 1 hour at 37° C. in round-bottom 96-well microtiter plates. Subsequently, irradiated (3000 rad) C57B1/6 (H-2^{b}) stimulator cells were added at a ratio of 2:1, the cultures were pulsed with 1 μCi/well [³H]thymidine, and the cells were harvested on day 5. Thymidine incorporation was measured using a liquid scintillation counter. For re-stimulation assays, secondary MLCs were established as is described above using 2×10⁷ spleen cells at a 1:1 responder:stimulator ratio in a 6-well culture plate. Cells were washed extensively on day 7, cultured for another 3 days in medium without mCTLA-4/Fc or mIgG2a, and then re-stimulated with irradiated C57B1/6 spleen cells. Cultures were then pulsed with 1 μCi/well [³H]thymidine, and aliquots were harvested daily on days 1 through 7. The level of [³H]thymidine incorporation was measured as is described above. Maximum [³H]thymidine incorporation was reached on days 2-3 (FIG. 6). In contrast, responder cells (DBA/2J) primed in the presence of mCTLA-4/Fc did not proliferate in response to reconfrontation with the original C57B1/6 strain stimulator cells (i.e., proliferation did not exceed 10% of the maximum proliferation of the positive control cultures at any time point).

Islet Cell Allograft Treatment with (NL) CTLA-4/Fc: To demonstrate that (NL) mCTLA-4/Fc could be incubated with grafts in vitro prior to transplantation to block B7-mediated rejection by donor tissues, crude islet cell isolates were harvested from DBA/2J mice by collagenase digestion and ficoll density gradient separation, as was previously described (Gloth et al., 1986, Transplantation 42:387). Approximately 300-400 islets per transplant were incubated at 37° C. for 1 hour with either media alone, control protein (mIgG3 at 10 μg/ml in RPMI), or (NL) mCTLA-4/Fc (at 10 μg/ml in RPMI). The cells were then pelleted and injected under the left renal capsule of B6AF1 recipients that had been rendered diabetic 7 days earlier by a single intraperitoneal injection of streptozotocin (225 mg/kg). The islet cell recipients were not systemically immunosuppressed. Graft function was monitored by tail blood glucose measurements using the Chemstrip bG and Accu-Chek III blood glucose monitor system (Boehringer Mannheim, Indianapolis, Ind.); other art-recognized methods of measuring blood glucose levels can also be used. Post-transplant primary graft function was defined by a blood glucose level of less than 11.1 mmol/L, and subsequent graft failure was defined by consistent blood glucose levels that were greater than 16.5 mmol/L. To detect graft tolerance, animals with functioning grafts were challenged after 120 days after transplantation with an intraperitoneal injection of 5×10⁷ irradiated (3000 Rad) donor splenocytes (Shizuru et al., 1987, Science 237:278).

All islet grafts (n=24) that were treated with (NL) mCTLA-4/Fc displayed signs of primary graft. function by the sixth day after transplantation. Of these 24 grafts, 10 (42%) went on to exhibit signs of long term (i.e., >150 days) engraftment (FIG. 7) in untreated allogeneic recipient hosts. In order to determine whether graft tolerance was achieved through treating the islet grafts with CTLA-4/Fc, hosts bearing long-term functioning islet grafts (i.e., >150 days) were challenged with donor spleen cells. Of these animals, 50% (3 out of 6) tolerated their grafts. In control experiments, islets were treated with mIgG3. Murine IgG3 proteins do not engage murine FcγRI, and they weakly activate complement as compared with mIgG2a isotypes (Paul, 1993, In: Fundamental Immunology, Raven Press). Moreover, IgG immunoglobulins only effect CDC activity as multimeric complexes, while monomeric IgG can bind Fc receptors (Burton, 1985, Molecular Immunology, 7:445). Therefore, a monoclonal mIgG3, which does not bind B7, was chosen as a control ligand for the (NL) mCTLA-4/Fc chimeric protein. All of the IgG3-treated islet grafts (n=9) demonstrated primary graft function, and 89% were acutely rejected (FIG. 7). Islets which were treated with medium alone (n=10) showed signs of primary graft function and were acutely rejected by day 44 (FIG. 7). Thus, only the CTLA-4/Fc-treated grafts were tolerated, and the incubation period of 1 hour for islet graft treatment in the presence of (NL) CTLA-4/Fc was sufficient to lead to significant engraftment.

Immunohistochemistry: The left kidney containing the islet cell graft of a tolerant animal (i.e., an animal in which the graft was functioning at 200 days after transplantation and at 50 days after donor spleen cell challenge) was removed and embedded in OCT compounds. Serial frozen sections were either fixed in cold acetone for immunocytochemistry or fixed in methanol for hematoxylin and eosin staining. Immunohistology was performed with conventional methods (see, e.g., Boegen et al., 1993, J. Immunol. 150(10):4197). Briefly, 0.3 μm sections were sequentially blocked with mouse serum, avidin, and biotin, then quenched with H₂O₂, and then incubated with rat anti-mouse CD4 or CD8 mAbs for 45 minutes in 0.05 M Tris buffer (pH 7.6) at room temperature. Binding of antibodies was detected with a biotinylated rabbit anti-rat mAb and avidin-HRPO complex, using diaminobenzidine for detection of enzyme activity. Negative controls were processed as above with the exclusion of the primary antibody. Sections were counter-stained with methyl green (C₂₇H₃₅BrClN₃.ZnCl₂).

Histologic analysis of islet cell allografts harvested from tolerant animals (i.e., >day 150 post transplantation and >day 50 post donor spleen cell challenge) demonstrated a dense molecular cell infiltrate surrounding, but not invading, the islets (FIGS. 8A-D). The majority of these cells were CD4⁺ cells; a significant number (approximately 30% of the level of CD4⁺ cells) of CD8⁺ cells were also detected. These data indicate that, while treatment of islet grafts with (NL) mCTLA-4/Fc does not eliminate cellular responses to the graft, the responding mononuclear cells do not aggressively infiltrate the islet tissue. Aggressive infiltration leads to islet cell destruction, and such infiltration is characteristic of rejection (see, e.g., O'Connell et al., 1993, J. Immunol. 150:1093).

What is claimed is:

1. A method for inhibiting rejection of a graft containing a cell which expresses a co-stimulatory protein in a patient, said method comprising treating said graft in said patient with a molecule which binds to a co-stimulatory protein of antigen-presenting cells to inhibit activation of host T-cells by said graft, wherein said molecule is a molecule other than lytic CTLA-4/Fc.

2. The method of claim 1, wherein said molecule is selected from the group consisting of CTLA-4, CD28, CD40L, and CD2.

3. The method of claim 1, wherein said graft is further treated ex vivo.

4. The method of claim 1, wherein said graft is further treated in a brain-dead, beating-heart donor.

5. The method of claim 1, wherein said co-stimulatory protein is selected from the group consisting of LFA-3, CD48, CD40, and B7 proteins.

6. The method of claim 5, wherein said co-stimulatory protein is a B7 protein selected from the group consisting of B7-1, B7-2, and B7-3.

7. The method of claim 1, wherein said molecule is a chimeric molecule comprising:
(i) a first polypeptide which binds to a co-stimulatory protein of antigen-presenting cells, and
(ii) a second polypeptide which is enzymatically inactive in humans and which increases the circulating half-life of said first polypeptide by a factor of at least two.

8. The method of claim 7, wherein said second polypeptide comprises albumin.

9. The method of claim 7, wherein said first polypeptide is selected from the group consisting of CTLA-4, CD28, CD40L, and CD2.

10. The method of claim 7, wherein said second polypeptide comprises the Fc region of an IgG molecule and said polypeptide lacks a variable region of an IgG heavy chain.

11. The method of claim 10, wherein said Fc region is lytic.

12. The method of claim 10, wherein said Fc region includes a mutation which inhibits complement fixation by said molecule.

13. The method of claim 10, wherein said Fc region includes a mutation which inhibits high affinity binding to the Fc receptor by said molecule.

14. The method of claim 7, wherein said enzymatically inactive polypeptide comprises an IgG hinge region.

15. The method of claim 7, wherein said enzymatically inactive polypeptide comprises a flexible polypeptide spacer.

16. The method of claim 1, wherein said graft is treated with a molecule comprising CD2 and with a molecule comprising CTLA-4.

17. The method of claim 16, wherein said treatment with said molecule comprising CTLA-4 occurs simultaneously with said treatment with said chimeric molecule comprising CD2.

18. A method for inhibiting rejection of a graft containing a cell which expresses a co-stimulatory protein in a patient, said method comprising treating said graft outside of said patient with a molecule which binds to a co-stimulatory protein of antigen-presenting cells to inhibit activation of host T-cells by said graft.

19. The method of claim 18, wherein said molecule is selected from the group consisting of CTLA-4, CD28, CD40L, and CD2.

20. The method of claim 18, wherein said molecule is a monoclonal antibody which specifically binds to a co-stimulatory protein of antigen-presenting cells.

21. The method of claim 20, wherein said co-stimulatory protein is selected from the group consisting of LFA-3, CD48, CD40, and B7 proteins.

22-44. (canceled)
